(19) United States
(12) Patent Application Publication
(10) Pub. No.: US 2004/0171975 A1
(43) Pub. Date: Sep. 2, 2004

(54) Casting aid and methods of forming casts

(76) Inventors: Roy A. Morris, Plantation, FL (US); Jose A. Alvarez, San Juan, PR (US); Eric R. Jania, Rockaway, NJ (US)

Correspondence Address: HARNESS, DICKEY & PIERCE, P.L.C., P.O. BOX 828, BLOOMFIELD HILLS, MI, 48303, US

(21) Appl. No.: 10/638,779

(22) Filed: Aug. 11, 2003

Related U.S. Application Data
(63) Continuation-in-part of application No. 09/775,308, filed on Feb. 1, 2001, now abandoned.

(51) Int. Cl. A61F 13/00

(52) U.S. Cl. 602/41

(57) ABSTRACT

Casting aids comprising a resin-impermeable substrate and a lubricating material, wherein the lubricating material is coated on at least one surface of said substrate. The casting aid may be used to deliver lubricant as needed to gloves or other surface contacting a tacky resin material during molding or forming of the tacky material. In particular, the lubricant-impregnated porous substrate may be used to lubricate gloves during molding and forming of orthopedic casting materials. The casting may be packaged as a kit with an orthopedic casting material.

### CROSS-REFERENCE TO RELATED APPLICATIONS

This application is a continuation-in-part of U.S. Pat. application Ser. No. 09/775,308 filed on Feb. 1, 2001. The disclosure of the above application is incorporated herein by reference.

### BACKGROUND OF THE INVENTION

The present invention relates to novel aids for use in forming orthopedic casts and other structures made of tacky resinous materials. In particular, this invention relates to flexible sheets, coated with a lubricant, that have no or limited permeability to the resin with which they are used. In a preferred application, the invention also provides methods for on-demand lubrication of gloves during the formation of orthopedic casts made using tacky resinous materials.

A wide variety of polyurethane, epoxy and other resins are known in the art for use in forming solid and semi-solid articles. In many instances it is necessary or desirable to form or mold such articles by hand. The resinous material may have a resinous matrix that may be filled with other materials, or may be a resin impregnated into a porous substrate. Practical uses of such materials include knitted or woven fabrics made of fiberglass or other materials impregnated with a tacky material that may be used to repair water pipes or boats, tacky epoxy materials that may be used in repairing mufflers and automobile bodies, and tacky epoxy resin wraps that may be used to repair poles such as utility poles.

Another area in which resinous materials are used, in particular, is in the formation of casts and splints for immobilizing a body member. Such casts and splints are used, for example, to protect a body part from injury, or to allow a broken bone to heal properly. Historically, such casts were made of plaster of paris. However, plaster casts have recently been largely replaced in many applications by orthopedic bandages or sheets impregnated with tacky synthetic resin mixtures. The synthetic resins used in combination with certain knitted or woven substrates offer a number of advantages over the old plaster materials. In particular, casts made from the synthetic materials are lighter, stronger, harden more quickly, allow for better air circulation, and are not water sensitive.

Among the synthetic resin compositions used to prepare casting materials are water-activated or water-curable synthetic resin compositions and thermoplastic resin compositions. Like the old plaster casting material, the water-activated or water-curable synthetic resin impregnated bandages or sheets, which may also be called “tapes,” are wetted before application to the body member. The water begins the curing reaction that hardens the cast. Other compositions for casting materials use synthetic resin compositions employing different kinds of curing mechanisms, for example crosslinking through unsaturation.

Gloves are often worn while molding such articles by hand, for aesthetic and safety concerns, so as to avoid direct contact of the skin with the resinous material. However, problems may arise due to the inherent tackiness of the resinous materials. For example, in applications involving resin-impregnated fabrics, tackiness of the resin may make it difficult to properly position the fabrics, or to smooth the fabric once in place. A variety of methods have been described in the art to address this problem.

For example, addition of lubricants or other materials to make the resin-impregnated casting sheets slippery is described in U.S. Pat. No. 4,667,661 Scholz et al., issued May 26, 1987, and U.S. Pat. No. 4,774,937 Scholz et al., issued Oct. 4, 1988. The surface of such sheets is said to have a “kinetic coefficient of friction” of less than 1.2. Other casting tapes having a lubricant are disclosed in U.S. Pat. No. 4,937,146, Dull et al., issued Jun. 26, 1990.

Alternatively, various means of delivering a lubricant to a glove used in forming the cast have been suggested. A mixture of water, sorbitol, mineral oil, and silicone fluid is commercially available from 3M Co., St. Paul, Minn., under the tradename Cast Cream. This product is applied to the gloves after wrapping, but before molding, the cast. Lubricated gloves are disclosed in European Patent Publication 712,618, Richard et al., published May 22, 1988; U.S. Pat. No. 5,438,709, Green et al., issued Aug. 8, 1985; and U.S. Pat. No. 5,439,439, Green et al., issued Aug. 8, 1995.

Other approaches are described in WO 94/23769, Scholz et al., which describes casting sheets separated by a water-soluble liner layer, which may include a lubricant. Porous sheets impregnated with a lubricant, which is then rubbed on the surface of a glove, are described in U.S. Pat. No. 5,925,004, Doubleday et al., issued Jul. 20, 1999.

However, such lubricating methods known in the art have disadvantages. In particular, methods involving pre-lubricated gloves or casting sheets offer no control over the amount of lubricant applied to the gloves, or when it is applied. More lubricant may be applied than is necessary or desirable. For example, it may be desirable to use some sheets with little or no lubricant, so as to have the wrapped layers stick to one another without slipping and to have the end of the sheet or bandage stick to the surface of the casting sheet wrap in order to terminate the application of the sheet. Moreover, incorporating certain lubricants, such as mineral oil, into the curable resin composition may result in longer set time due to the dilution of the resin curing sites. It is also preferable to avoid the transfer of lubricant to the casting resin during storage.

### SUMMARY OF THE INVENTION

The present invention provides improved methods and articles for delivering a desired amount of lubricant to selected sites on the gloves one or more times when working with tacky materials, in particular tacky resins and polymers. Such methods and articles include, in particular, casting aids and methods for making resinous orthopedic casts. Such casting aids comprise a resin-impermeable substrate, and a lubricating material, wherein said lubricating material is coated on at least one surface of said substrate. In one embodiment, the substrate is a non-porous flexible sheet comprising a thermoplastic polymer. In another embodiment, the substrate is a slightly porous fabric, which is resin impermeable. The lubricating material is preferably a hydrophilic or water-soluble polymer, such as polyvinylpyrrolidone.

The present invention also provides kits and methods for the formation of an orthopedic cast. Kits comprise a casting material and a casting aid comprising a resin-impermeable substrate and a lubricating material coated on a surface of said substrate. Methods use a casting material, a casting aid, and a glove, and comprise the steps of:

(1) transferring the lubricating material to a surface of the glove;

(2) applying the casting material to a body member using the glove; and

(3) allowing the casting material to harden to form an orthopedic cast.

Applicants have found that the methods and articles of this invention provide benefits versus methods and articles among those in the art. Such benefits include one or more of enhanced lubrication, greater control of the amount of lubricant delivered, greater control over the time of lubricant use, enhanced lubrication using lesser amounts of lubricant, lower levels of lubricant in the composition, and control or elimination of the exposure of the resin to lubricant when the casting aid is packaged with the resin material. It should be understood that the description and specific examples, while indicating embodiments of the invention, are intended for purposes of illustration only and are not intended to limit the scope of the invention. Moreover, the description of embodiments having certain recited features is not intended to exclude other embodiments having additional features, or other embodiments incorporating different combinations of the recited features.

### BRIEF DESCRIPTION OF THE DRAWINGS

FIG. 1 is a depiction of a device useful in measuring the permeability of substrates for use in the compositions of this invention.

It should be noted that the figure set forth herein is intended to exemplify the general characteristics of a device among those useful in the practice of this invention. This figure may not precisely reflect the characteristics of any given embodiment, and is not necessarily intended to define or limit specific embodiments within the scope of this invention.

### DETAILED DESCRIPTION

The invention provides an article that comprises a resin-impermeable substrate coated with a lubricant. The coated substrate is used to lubricate a glove or other resin handling article (herein referred to as a “glove”) before handling tacky resin materials, so that the tacky material may be worked with and formed without sticking to the glove. Articles made using such tacky resin materials (herein “resinous articles”) include any of a wide variety of articles which are formed in whole or in part by the application of a resin to a substrate or form. A preferred resinous article is an orthopedic cast.

As referred to herein, an “orthopedic cast” is a device which encloses, in whole or in part, a body member (e.g., a leg) of a human or other animal subject. Such casts may be flexible, semi-rigid, or rigid, and may be used for the prevention of injury, or in the treatment of disorders such as broken bones. Specific compounds, compositions and other components to be used in such orthopedic casts must, accordingly, be pharmaceutically and cosmetically acceptable. As used herein, such a “pharmaceutically and cosmetically acceptable” component is one that is suitable for use with humans and/or animals without undue adverse side effects (such as toxicity, irritation, and allergic response) commensurate with a reasonable benefit/risk ratio.

### Casting Aids

The present invention provides casting aids comprising a resin-impermeable substrate, and a lubricating material, wherein said lubricating material is coated on at least one surface of said substrate. As referred to herein, a “casting aid” is an article that is useful in the handling of resinous materials, or formation of articles comprising resinous materials. Preferred articles comprising resinous materials include orthopedic casts. (As used herein, the word “include” is intended to be non-limiting, such that items that are included in a list are not to the exclusion of other like items that may be useful in the compositions and methods of this invention.)

Substrate:

As referred to herein, a “resin-impermeable substrate” is a material that is not chemically reactive with, and is substantially impermeable to, the resin with which it is to be used. A “substantially impermeable” substrate, having two faces, is a material that prevents significant contact between lubricant coated on one face when the other face is placed in contact with a resinous material. In embodiments in which a casting aid is packaged with the resinous material, the substrate prevents substantial contact between the resin and the lubricating material that is coated on the substrate.

In one embodiment, the substrate is impermeable to viscous fluids. In another embodiment, the substrate has low permeability to viscous fluid. As referred to herein, in a preferred embodiment, “permeability” of a substrate refers to the Permeation Value of the substrate, as determined using a Permeability Test described herein.

Permeability Test:

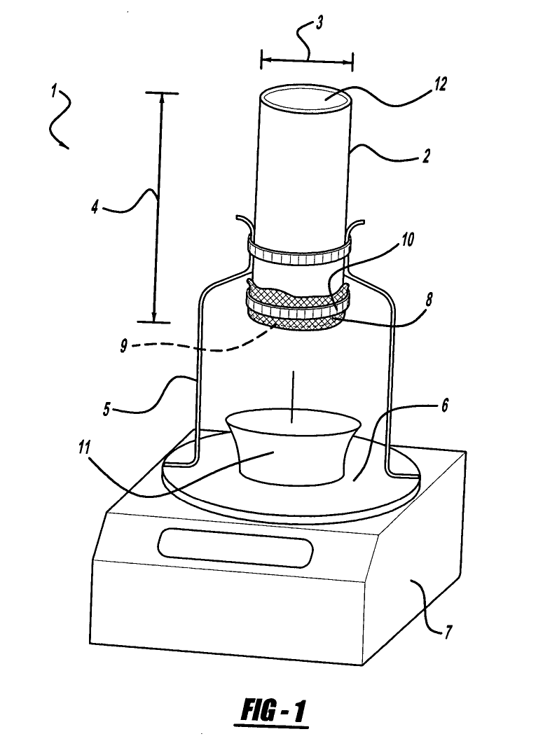

The Permeation Value of a substrate can be determined using a Permeability Device generally depicted in FIG. 1. As shown in FIG. 1, the Permeability Device (1) comprises a cylinder (2) which has a diameter (3) of about 2 inches (5.1 cm). Preferably, the cylinder has a length (4) of about 12 inches (30.5 cm). The cylinder can be formed of any suitable material, such as polyvinyl chloride, that is not reactive with the viscous fluid used in the Permeability Test. The cylinder is supported, by three or more support members (5, only two of which are shown), so that the cylinder is essentially vertical. The sample substrate (8) is secured over the lower open end (9) of the cylinder, using one or more clamps (10). The substrate is secured so that it is substantially taut, but not stretched, over the cylinder opening, such that a single layer of the substrate covers the entire open end of the cylinder, without wrinkles. A tared collection cup (11) is placed directly under the cylinder (2).

In the Permeability Test, an oil having a viscosity of about 5200 centistokes at 40° C. is used as the viscous liquid. A suitable liquid is sold as STP® Smoke Treatment, by the STP Products Company, Brookfield, Connecticut, U.S.A. The Permeability Device and a tared collection cup (11) are placed on the platen (6) of a conventional laboratory scale (7) and weighed. The collection cup (11) is placed directly under the cylinder (2). Under ambient conditions (approximately 21° C.), liquid is then poured into the upper open end (12) of the cylinder (2), until the weight of the Permeability Device and collection cup are increased by 114 grams (i.e., 144 grams of liquid are poured into the cylinder). The liquid is poured quickly, taking care to pour the liquid directly onto the substrate (8) and not on the walls of the cylinder. A stop watch is started upon initiation of pouring, and 15 minutes is allowed to pass. After lapse of 15 minutes, flow of the liquid into the cup is stopped, e.g., by placing a cup or other containment device under the cylinder (2) to intercept the flow of liquid, followed by removal of the Permeability Device (1) from the scales. The weight of the collection cup is then taken, and the weight of collected oil is determined by subtracting the tare weight of the cup. The Permeation Value is then calculated as weight/minute, by dividing the weight of the collected oil by 15 minutes.

Preferably the resin impermeable substrate has a Permeation Value of less than about 2.8, preferably less than about 2.5, more preferably less than about 2.3. In one embodiment, the Permeation Value is from about 2.0 to about 2.3, preferably about 2.2. In another embodiment, the Permeation Value is less than about 2.0, preferably less than about 1.5, preferably less than about 1.0, preferably less than about 0.5. In one embodiment, the Permeation Value is about zero.

Preferably, the substrate has at least some flexibility or deformability that aids in depositing the lubricating material on the glove or other article that is to be lubricated. The substrate may have a broad range of sizes, but preferred substrates are of a size that is large enough to deliver a sufficient amount of lubricant but small enough to be easily handled. Preferably the substrate is a sheet from about 0.25 mm (1 mil) to about 1.0 mm (4 mil), more preferably about 0.5 mm (2 mil), thick. Preferably the substrate sheet is from about 1 cm (.4 in) to about 50 cm (19.6 in) in length, more preferably from about 3 cm (1.2 in) to about 38 cm (15 in) length, more preferably from about 15 cm (5.9 in) to about 25 cm (9.8) in length. Preferably the substrate sheet is from about 1 cm (0.4 in) to about 50 cm (19.6 in) in width, more preferably from about 2 cm (0.78 in) to about 25 cm (8.8 in) in width, more preferably from about 5 cm (1.8 in) to about 13 cm (5.1 in) in width. The substrate may be an individual sheet, or may be in the form of a continuous web of sheets connected in an end-to-end relationship separated by a scored line or perforations, much like paper toweling.

In one embodiment, the substrate comprises a thermoplastic polymer. Preferred polymers include polyolefins (such as polypropylene), polyesters, polymethylpentene, polyetheretherketone, polyvinyl chloride, polyvinylidene fluoride, polycarbonates, acrylics and acrylic copolymers (such as polyacrylonitrile), nylon, fiberglass, and mixtures thereof. Particularly preferred polymers include polyesters and polyolefins, particularly polyester high and low density polyethylene and polybutylene. Preferably, polyolefin and polyester films are oxidized using, for example, a high-voltage corona discharge. A preferred substrate comprises polyester.

In one embodiment, the substrate is a non-porous film. A particularly preferred substrate is a Mylar® polyester film, manufactured by E. I. duPont de Nemours and Company.

In another embodiment, the substrate is a slightly porous fabric. As used herein, the term “slightly porous” refers to a substrate that comprises pores, and which is permeable to air or water, but which is substantially impermeable to the resin with which it is to be used. A preferred slightly porous fabric comprises polyester, preferably a polyester apparel fabric. Preferably the slightly porous fabric has greater than about 50 courses (threads) per square inch. In various embodiments, the slightly porous fabric has from about 60 to about 90, from about 70 to about 90, or from about 80 to 85, courses per square inch.

Lubricating Material:

The substrate is coated with a lubricating material. As used herein, “coated” refers to any process by which lubricant material is deposited on or into the surface of the substrate, preferably resulting in a uniform layer of lubricating material, so that lubricating material may be readily transferred to a glove or hand during use.

Lubricating materials useful herein comprise a lubricant that can be transferred from the substrate to a glove, and that reduces friction between a glove and the resinous article that is to be formed using the glove. Such lubricating materials preferably do not significantly react with the substrate, glove, or resin used to make the resinous article. Lubricants useful in the lubricating materials of this invention include film forming lubricants, preferably film forming hydrophilic polymers. Lubricants useful herein include polyethylene oxide; polyethylene oxide block copolymers such as copolymers of ethylene oxide and propylene oxide; polyvinyl alcohol; hydroxyethyl cellulose; carboxymethyl cellulose; acrylamide-based polymers; polyvinylpyrrolidone; lecithin-based lubricants; sulfonated or carboxylated polymers, such as sulfonated or carboxylated polyurethanes; hydrophilic, oligomeric diols, such as the reaction product of polyethylene oxide glycol with dimethyl sodium sulfoisophthalate in a 2:1 molar ratio; hydrophobic lubricants and oils, such as mineral oils, petrolatum, vegetable oils, and derivatives thereof; synthetic and natural motor oils; silicones and other fluids, oils, and greases, such as polydimethylsiloxanes, polymethylphenylsiloxanes, and polydiphenylsiloxanes, especially those having viscosities of between about 100 and 100,000 centistokes; fluorinated greases; and mixtures thereof. Preferred lubricants include hydrophilic polymers, such as hydroxyethyl cellulose, carboxy methyl cellulose, polyvinyl alcohol, polyvinylpyrrolidone, polyacrylic acid salts, polyethylene oxide homopolymers and copolymers, and mixtures thereof. In embodiments of this invention comprising a slightly porous fabric, preferred lubricants comprise polyvinyl alcohol, polyvinylpyrrolidone, polyacrylic acid salts, polyethylene oxide homopolymers and copolymers, and mixtures thereof. Polyvinylpyrrolidone is a particularly preferred lubricant.

The lubricating material preferably also comprises a surfactant. Surfactants useful in compositions of the present invention include anionic, nonionic, cationic, zwitterionic and amphoteric surfactants. Anionic surfactants include alkyl and alkyl ether sulfates; the water-soluble salts of organic, sulfuric acid reaction products such as alkali metal and ammonium sulfonated C₁₂₋₁₈ n-paraffins; reaction products of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil; succinamates, such as disodium N-octadecylsulfosuccinamate, tetrasodium N-(1,2-dicarboxyethyl)-N-octadecylsulfosuccinamate, diamyl ester of sodium sulfosuccinic acid, dihexyl ester of sodium sulfosuc cinicacid, and dioctyl esters of sodium sulfosuccinic acid; and olefin sulfonates having about 12 to about 24 carbon atoms. Nonionic surfactants include those produced by the condensation of alkylene oxide groups (hydrophilic in nature) with an organic hydrophobic compound, which may be aliphatic or alkyl aromatic in nature. Examples of preferred classes of nonionic surfactants are condensation products of ethylene oxide with the product resulting from the reaction of propylene oxide and ethylene diamine products; condensation products of aliphatic alcohols having from about 8 to about 18 carbon atoms, in either straight chain or branched chain configuration, with ethylene oxide; long chain tertiary amine oxides; long chain tertiary phosphine oxides; and long chain dialkyl sulfoxides containing one short chain alkyl or hydroxy alkyl radical of from about 1 to about 3 carbon atoms (usually methyl) and one long hydrophobic chain which include alkyl, such as octadecyl methyl sulfoxide, 2-ketotridecyl methyl sulfoxide, 3,6,9,-trixaoctadecyl 2-hydroxyethyl sulfoxide, dodecyl methyl sulfoxide, oleyl 3-hydroxypropyl sulfoxide, tetra decyl methyl sulfoxide, 3-methoxytridecyl methyl sulfoxide, 3-hydroxytridecyl methyl sulfoxide, and 3-hydroxy-4-dodecoxybutyl methyl sulfoxide. Cationic surfactants useful in compositions of the present invention include those comprising amino or quaternary ammonium hydrophilic moieties that are positively charged when dissolved in the aqueous composition of the present invention. Among the quaternary ammonium-containing cationic surfactant materials useful herein are diquaternary ammonium salts, such as tallow propane diammonium dichloride; dialkyldimethyl-ammonium chlorides, wherein the alkyl groups have from about 12 to about 22 carbon atoms and are derived from long-chain fatty acids, such as hydrogenated tallow fatty acid; and salts of primary, secondary and tertiary fatty amines, such as stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (5 moles E.O.) stearylamine, dihydroxy ethyl stearylamine, and arachidylbehenylamine. Zwitterionic surfactants useful herein are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic water-solubilizing group, e.g., carboxy, sulfonate, sulfate, phosphate, or phosphonate. Other zwitterionics such as betaines are also useful in the present invention. Examples of amphoteric surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Surfactants among those useful herein are disclosed in the following documents, all incorporated by reference herein: McCutcheon's, Detergents and Emulsifiers, 1984 Annual, published by Allured Publishing Corporation, Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr., issued Jun. 7, 1983; and U.S. Pat. No. 4,275,055, Nachtigal et al., issued Jun. 23, 1981. In one embodiment, the lubricant combines a mixture of polyvinylpyrrolidone and alkyl sulfosuccinate anionic surfactant. In another embodiment, the lubricant consists essentially of polyvinylpyrrolidone.

The lubricating materials are preferably aqueous solutions, comprising from about 5% to about 80%, preferably from about 10% to about 30%, more preferably from about 10% to about 20% of the lubricant, depending on the lubricant. (All percentages herein are by weight, unless otherwise specified.) The lubricant material may include other ingredients, such as stabilizers, thickening agents, pigments or colorants, dyes, organic solvents and cosolvents. Preferably the lubricating material contains an antiblocking agent, to facilitate rolling the casting aid by preventing lubricant layers from sticking together. Preferred antiblocking agents include particulates having a particle size of from about 10 microns to about 400 microns, comprising mineral fillers such as talc and calcium carbonate, ceramic microspheres, aluminum or other oxide powders, polymers such as polystyrene, and glass microspheres.

The lubricating material may be coated on the substrate in any fashion that allows the lubricant to be transferred from the substrate to the glove when in use. Preferably, the lubricating material forms an essentially uniform layer covering substantially all of at least one surface of the substrate. In preferred embodiments wherein the substrate is a flexible sheet, the lubricating material can be coated on either or both of the major surfaces of the sheet. In a preferred embodiment, the lubricating material is coated on only one of the major surfaces of the sheet. Preferably the lubricating material is coated at a level of from about 0.002 to about 0.005 g/cm². More preferably, the lubricating material is coated at a level of from about 0.003 to about 0.004 g/cm². (As referred to herein, “g/cm²” is the weight, in grams, of the lubricating material per square centimeter of substrate surface.)

After coating, the casting aid is preferably dried to remove substantially all moisture. In an embodiment wherein the lubricant comprises polyvinylpyrrolidone, preferably the drying is conducted at a temperature of from about 65° C. to about 120° C. In one embodiment, the drying temperature is from about 90° C. to about 115° C., preferably from about 100° C. to about 115° C. In another embodiment, the drying temperature is from about 65° C. to about 100° C., preferably from about 60° C. to about 80° C. Preferably the substrate is coated so that the relative dry weight percent of the coating (the weight of coating, after drying, as a percentage of the weight of substrate) is from about 80% to about 300%. Preferably, the relative dry weight of the coating is from about 100% to about 125%, preferably from about 110% to about 120%.

The casting aid may be packaged, with one or more article per package or container, for storage before use. The invention further provides a container comprising one or more of the casting aids. In the case of a volatile lubricant, the container may comprise a closed or, preferably sealed, package capable of preventing evaporation of the lubricating material, wherein the package contains one or more of the casting aids. Suitable packaging is well known in the art. For example, a package dispenser for dispensing sheets of the invention may comprise a container having a cavity containing a plurality of the sheets. The container may have an opening on one end through which said sheets may be taken or pulled. The container may have one or more leak-proof layers, such as a hermetic seal. The seal may be made of a flexible material or of a deformable plastic coated foil or uncoated foil. The container may be made of either a rigid or a flexible material, or a combination of rigid and flexible materials. For example, the container may be formed from a rigid plastic or semi-rigid plastic such as a polyethylene, polypropylene, polyethylene terephthalate, or from a more flexible material such as a composite film.

Multiple casting aids may be placed within the container in random or ordered configurations. Accordingly, the sheets may be ordered in stacks or rolls in the containers. The container may contain, for example, a continuous web of material in compacted form having sheets connected in end-to-end relationship separated by scored lines or perforations, from which the respective sheets can be readily dispensed, one at a time. Such a container may include a body containing the continuous web; a closure for the container; and a dispensing opening through which the web is withdrawn and a sheet of the web separated at the scored line or perforations. Preferably, as each sheet is pulled out and separated, the next sheet is exposed above the dispensing opening. The web may be in the form of a roll, and the web may be drawn for dispensing from the center of the roll. Alternatively, the sheets may be stacked face-to-face in the container. In yet another embodiment, the sheets may be partially overlapped or stacked as an interfolded array so that when one is withdrawn from the container, the next in the stack is presented in a graspable position.

### Kits

The present invention also provides kits for the formation of an orthopedic cast, comprising a casting material and a casting aid. As used herein, a “casting material” is any material which comprises a curable resin for use in making an orthopedic cast. The curable resin may be any of the kind known in the art, including water-curable, UV-curable, EB-curable, heat-setting, and air-dry (oxygen-curing) materials. The casting material is preferably a curable-resin impregnated sheet (herein, a “tape”), preferably impregnated with a water-curable resin.

The curable resin of the casting material may be any curable resin that satisfies the functional requirements of an orthopedic cast. The resin-impregnated tape should be pliable before cure, but should upon cure become rigid and strong to support the loads and stresses to which the cast may be subjected by the activities of the patient. Such casts may be rigid, semi-rigid, or flexible, depending upon the nature of the condition to be treated.

Curing of the resin should take place fairly rapidly once curing has been initiated, preferably attaining weight bearing strength (for those casts that intended to be rigid) within about 30 minutes. The curable resin is selected from materials that preferably do not emit significant amounts of toxic by-products during curing that might be harmful to either the patient or the person applying the cast. The curable resin also is preferably non-irritating to the skin and should not generate an excessive amount of heat during curing that might cause discomfort or burns to the underlying skin.

The curable resin may cure by a variety of mechanisms. The cure reaction should not generate an amount of heat that would cause discomfort or burns to exposed skin. Curable resin systems useful herein include catalyzed polyurethane prepolymers and water-reactive alkoxysilane terminated resins.

The curable resin is preferably a polyurethane prepolymer. Useful polyurethane prepolymers are described, for example, in U.S. Pat. No. 4,131,114 Kirkpatrick et al., issued Dec. 26, 1978; U.S. Pat. No. 4,376,438, Straube et al., issued Mar. 15, 1983; U.S. Pat. No. 4,411,262 von Bonin et al., issued Oct. 25, 1983; U.S. Pat. No. 4,968,542 Gasper et al., issued Nov. 6, 1990; and U.S. Pat. No. 4,502,479 Garwood et al., issued Mar. 5, 1986; incorporated by reference herein. The polyurethane prepolymer comprise, and may be prepared by reacting, one or more polyisocyanates with one or more polyols in an isocyanate/hydroxyl equivalent ratio of from about 2:1 to about 12:1, preferably from about 3:1 to about 5:1. The polyisocyanate may be aliphatic, cycloaliphatic, or aromatic diisocyanates, triisocyanates, or tetraisocyanates, as well as biurets, isocyanurates, and similar oligomers of these. Examples of useful polyisocyanates include, without limitation, toluene diisocyanates (TDI), including the 2,4 and 2,6 isomers and mixtures of these isomers; diphenylmethane diisocyanates (MDI), including the 4,4′, 2,4′, and 2,2′ isomers and mixtures of these isomers; hydrogenated diphenylmethane diisocyanates, aromatic polyisocyanates derived from phosgenation of the condensation product of aniline and formaldehyde (polymeric MDI), hexamethylene diisocyanate, isophorone diisocyanate, octamethylene diisocyanate, trimethylhexane diisocyanates, dodecamethylene diisocyanates, cyclopentane diisocyanate, cyclohexane diisocyanate, tetramethylxylene diisocyanate, and biurets, allophonates, isocyanurates, and substituted derivatives of these, such as carbodiimide-containing polyisocyanates. Preferred among these are polymeric diphenylmethane diisocyanates (polymeric MDI's).

The polyisocyanate is reacted with at least one polyol. Examples of suitable polyols include, without limitation, polyether polyols, polyester polyols including polycaprolactone polyols, and monomeric diols and triols such as 1,6-hexanediol. Preferred polyols include polyethylene oxide and polypropylene oxide diols and triols, polytetramethylene ether glycols, especially those having a molecular weight of from about 400 to about 4000. Polyalkylene oxide polyols are available commercially, for example from BASF Corporation under the tradename Pluracol® and from Union Carbide Corporation under the tradenames Carbowax® and Polyox®.

Preferred polyols include polyethylene oxide and polypropylene oxide diols and triols, having a molecular weight of at least about 2,000, preferably from about 2,000 to about 4,000, more preferably from about 3,000 to about 4,000. Preferably the polyol has a hydroxyl number of from about 28 to about 56, preferably from about 28 to about 46 more preferably from about 35 to about 40. As referred to herein, the “hydroxyl number” is the number of milligrams, per gram of polyol, of potassium hydroxide having an acid neutralization capacity equal to the polyol. Preferred polyols include PPG 3025, having a molecular weight of about 3,000 and a hydroxyl number of from about 35 to about 40, sold by Lyondell Chemical Company, and QO Polymeg 3000, having a molecular weight of from about 2,800 to about 3,000 and a hydroxyl number of from about 37 to about 40, sold by Great Lakes Chemical Corporation.

The curable resins of this invention preferably have a level of free NCO of from about 4% to about 10%, preferably from about 6% to about 10%, preferably from about 7% to 10%, preferably from about 7% to about 8%, more preferably about 7.5%. As referred to herein, the “level of free NCO” is the weight of the equivalents of NCO in excess of the equivalents of polyol in the prepolymer, as a percentage of the total weight of the prepolymer (weight of isocyanate plus the weight of the polyol). Preferred curable resins are disclosed in co-filed U.S. Patent Publication 2002/0160684, Morris et al., published Oct. 31, 2002, incorporated by reference herein.

The prepolymer is preferably mixed with a catalyst. Preferred catalysts include tertiary amine catalysts such as tertiary alkanolamines, for example dimethylethanolamine and dimethylaminodiethyl ether; 2,2′-dimorpholinodialkylethers such as 2,2′-dimorpholinodiethylether (DMDEE), available commercially from Texaco, Inc., as Thancat DMDEE; and 2,2′-dimorpholinyldialkylethers, such as 4-[2-[methyl-2-(4-morpholinyl)ethoxy]-ethyl]morpholine (MEMPE). The catalyst is preferably included in amounts of from about 0.1% to about 10% by weight of the impregnating resin mixture.

Preferably the casting material is substantially free of lubricating materials. As used herein, such “lubricating materials” are those disclosed in the art for admixture with the resin so as to make the resin slippery. Such lubricating materials are disclosed, for example, in U.S. Pat. No. 4,667,661, Scholz et al., issued May 26, 1987; and U.S. Pat. No. 4,774,937, Scholz et al., issued Oct. 4, 1988; both of which are incorporated by reference herein. As referred to herein, casting materials that are “substantially free” of lubricating materials have no lubricating materials, or levels of lubricating materials that are sufficiently low so as to provide insignificant reduction in the tackiness of the resin. Preferably the casting materials contain less than 1% of such lubricating materials.

The curable resin mixture preferably has a viscosity that is low enough to allow the mixture to enter the pores of the tape, while the viscosity is high enough that a sufficient amount remains in the tape so that upon cure the tape is effectively hardened and the desired physical properties are obtained. The viscosity may be modified according to a variety of means, including those known in the art. Such means include addition of low viscosity materials such as organic solvents to reduce viscosity, or by addition of thickening agents to increase viscosity. The optimum viscosity may be determined by straightforward testing. In a preferred embodiment of the invention, the viscosity of the curable resin mixture is from about 5000 to about 100,000 centipoise. The impregnating resin mixture may include other ingredients, such as stabilizers, thickening agents, antifoam agents, pigments, and colorants.

The tape is preferably an open-weave structure of a fibrous material. Examples of suitable porous material for the casting sheet include, without limitation, woven, knit, and non-woven fabrics of natural and/or synthetic fibers. Fabrics among those useful herein are described in U.S. Pat. No. 3,686,725, Nisbet et al., issued Aug. 29, 1972; U.S. Pat. No. 3,787,272, Nisbet et al. issued Jan. 22, 1974; U.S. Pat. No. 4,323,061, Usukura, issued Apr. 6, 1982; U.S. Pat. No. 4,609,578, Reed, issued Feb. 28, 1984; U.S. Pat. No. 4,668,563, Buese et al., issued May 26, 1987; and U.S. Pat. No. 4,745,912, McMurray, issued May 24, 1988; all of which are incorporated by reference herein. A preferred embodiment employs a knitted fabric that combines a high modulus fiber, such as a fiberglass, polyaramide, or carbon fiber, with an elastomeric, highly extensible fiber, such as a natural rubber, spandex (a polyurethane), polyisoprene, polyethylene, polypropylene, polybutadiene, diene copolymers, acrylonitrile copolymers, EPM, or EPDM fiber. A particularly preferred tape is comprised of fiberglass and polypropylene, preferably comprising from about 8% to about 18%, more preferably from about 10% to about 14%, of polypropylene.

The tape may be coated with a substance for modifying the fibers of the tape prior to impregnation with the curable resin mixture. The modifying substance preferably does not interfere with the impregnation or detrimentally affect the performance of the curable resin mixture. One example of a modifying substance is a low modulus binder that may be applied to prevent or reduce fraying of cut ends of the substrate, as is disclosed in U.S. Pat. No. 4,800,872, Buese et al., issued Jan. 31, 1989 incorporated by reference herein.

The tape, when impregnated with the curable resin, preferably has at least some flexibility. The impregnated casting material should have sufficient flexibility so that it can be molded about a limb without excessive pressure to support and/or protect the limb. The casting material is at least partially impregnated with a curable resin. Preferably, the resin mixture may flow into the capillary spaces between fibers of the fabric. Also preferably, the curable resin mixture that is impregnated into the tape comprises from about 25% to about 60%, preferably from about 30% to about 50%, preferably from at 35% to about 48%, more preferably from about 40% to about 45%, by weight of the impregnated tape.

The specific composition of the casting aid need not depend upon the curing mechanism of the tacky material. However, preferably the lubricating material is selected so as to avoid undesired interactions with the resin mixture. Accordingly, in one embodiment, the lubricating material preferably does not comprise a hydroxyl-functional lubricant.

The kits of this invention preferably comprise the casting aid and casting material in a suitable package. When the cast material is a thermoplastic, water-curable, or air-drying formulation, it is preferred for the casting material to be sealed in a compartment of the kit. The casting aid may be sealed in the same compartment as the casting material, or in a different compartment which may be sealed or unsealed. In a preferred embodiment, the casting material comprises a roll of sheets, around the circumference of which the casting aid is wrapped. Accordingly, such casting compositions of this invention comprise:

(a) a casting material formed into a roll; and

(b) a casting aid wrapped around the circumference of said roll.

In another preferred embodiment, the casting aid and the casting tape are folded into a single roll, the roll being then sealed inside of a compartment of the kit. In yet a further embodiment of the invention, the casting aid the casting tape are separated by a third layer, all three layers being folded into a single roll and the roll being then sealed inside of a compartment of the kit.

The casting tape that is rolled into the roll of the kit may be from about 2.7 m (3 yards) to about 4.1 m (4.5 yards), preferably from about 3.6 m (4 yards) to about 3.8 m (4.2 yards) in length and from about 2.5 cm (1 inch) to about 25.4 cm (10 inches), preferably from about 2.5 cm (1 inch) to about 12.7 cm (5 inches), in width. Preferably the casting tape is wrapped around a cylindrical core, preferably cylindrical core described in U.S. Pat. No. 5,984,884, Alvarez et al., issued Nov. 16, 1999 (incorporated by reference herein). Such cylinders preferably have multiple “L”-shaped projections that extend radially outward from the core of the cylinders.

The sealing compartments may comprise a peelable layer over a shell of a material inert toward both the lubricant-impregnated substrate and the water-curable resin-impregnated cast sheet. Alternatively, the kit may comprise foil, foil-lined, plastic, or metallized plastic pouches or compartments.

The kit may further include gloves suitable for wearing during the cast molding process. Suitable gloves that may be used to handle the tacky material include gloves made from vinyl (such as polyvinyl chloride), latex, butyl rubber, or other such elastomeric materials. The gloves should be substantially nonporous to the lubricant so that the lubricant is not absorbed by the glove but instead remains, at least for the most part, on the surface of the glove.

### Methods of Making Casts

The present invention also provides methods for forming an orthopedic cast by hand around a body member of a human or other animal subject, using a casting material, a casting aid, and a glove. Such methods comprise the steps of:

(1) transferring the lubricating material to a surface of the glove;

(2) applying the casting material to a body member using the glove; and

(3) allowing the casting material to harden to form an orthopedic cast.

The step of transferring the lubricating material to the glove is performed by contacting a surface of the glove with the casting aid, for example by rubbing the casting aid on the desired surface of the glove one or more times. One or two gloves may be used during the methods of this invention. One or both of the casting aid or the glove may optionally be wetted with water or immersed in water before transferring the lubricant to the glove, and this step may be preferred when the lubricant is water soluble or water dispersible. In some cases, the casting aid may be immersed in water or wetted before being used one or more times to deliver lubricant as needed to the gloves.

In the step of applying the casting material to a body member, the casting tape is wrapped about the subject's body member, or otherwise applied in multiple adjacent layers, using the glove(s) to which the lubricant has been applied by means of the casting aid. The application is assisted by the reduction in tackiness of the substrate relative to the gloves without reducing significantly the tackiness of the cast substrate relative to itself. When the desired amount of casting material has been wrapped around the body member, it is often desirable to further mold the wrapped cast material about the body member. Lubricant may be re-applied from the casting aid to the gloves before and during molding the wrapped cast material as desired.

An important aspect of many of the preferred embodiments of the present invention is the ability of the person applying the casting material to control the amount of lubricant applied to the gloves during molding of the cast, as well as the timing of the application. If the gloves begin to stick to the cast tape during the step of applying the tape, lubricant may be reapplied to the glove(s) by wiping the glove(s) with the casting aid. Accordingly, preferred methods of this invention comprise an additional step of transferring the lubricating material to a surface of the glove performed during the step of applying the casting material to the body member.

It is also sometimes desirable to use less lubricant prior to and during application of the casting material, so that the tape and packaging can be easily handled during the wrapping step. This can be accomplished by limiting the transfer of lubricant from the casting aid to the glove(s).

After application of the casting material is completed, the casting material is allowed to fully harden to form a cast about the wrapped limb. In one embodiment of the invention, the casting material comprises a thermoplastic composition that hardens on cooling. In a preferred embodiment of the invention, the casting material comprises a curable composition-impregnated sheet, particularly a water-reactive or water-activated curable composition. In the case of a water-reactive or water-activated curable composition, the casting material is immersed in water or otherwise wetted with water prior to applying the casting material around the body member. After the casting material is applied, preferably by wrapping and molding about the body member, the curable composition is cured to form the finished cast.

The following non-limiting examples illustrate the compositions and methods of the present invention.

### EXAMPLE 1

A casting material is made comprising a casting tape of fiberglass with approximately 10% polypropylene fiber, about 3 inches (7.6 cm) wide, 4 yards (3.7 m) long, and 1 mm (4 mil) thick. The tape has approximately 25% stretch and a mesh size of approximately 43.4 holes/cm² (280 holes/inch²). A 230-meter (250-yard) roll of casting tape is partially fused by applying a narrow band of heat at approximately 315° C. (600° F.), at 3.7-meter (4-yard) intervals. The roll is then dried at approximately 110° C. (230° F.) for approximately 4 hours.

A resin composition is made as having the following composition:

| Material | % (by weight) |
|---|---|
| Isonate ™ 2143L¹ | 58.0 |
| PPG 725² | 38.0 |
| dimorpholinodiethyl ether | 2.4 |
| benzoyl chloride | 0.1 |
| silicone 200 fluid³ | 0.3 |

¹diphenylmethane diisocyanate resin, sold by Dow Chemical, Midland, Michigan
²a polypropylene glycol, sold by Arco Chemical Co., Charlotte, North Carolina
³35,000 centipoise polydimethylsiloxane, sold by Dow Corning Corp., Midland, Michigan

The composition is made by admixture of the components, and heated with stirring to approximately 71° C. (160° F.) for approximately 4 hours. The roll of casting tape is removed from the drying oven and passed through the resin. After passing through the resin, the tape is squeezed through rollers to remove excess resin, so that the resin is impregnated at a level of about 42% by weight of the final impregnated tape. The tape is then cut where fused, to form individual strips of tape that are 3.7 m (4 yards) in length. An individual tape is then wound on a cylindrical core described in U.S. Pat. No. 5,984,884, Alvarez et al., issued Nov. 16, 1999 (incorporated by reference herein).

A casting aid according to this invention is made having the following lubricating material composition.

| Material | % (by weight) |
|---|---|
| carboxymethylcellulose | 13.6 |
| sodium dihexyl sulfosuccinate | 9.5 |
| talc | 20.0 |
| water | 56.9 |

The lubricating material is coated on a sheet of surface-oxidized Mylar® polyethylene film approximately 10.2 cm wide (4 inches), 20.3 cm (8 inches) long, and 0.8 mm (3 mil) thick. The coated film is then dried in an oven, resulting in a coating layer approximately 0.34 mm (1.5 mils) thick. The casting aid is then wrapped around the roll of impregnated casting sheet. The wrapped roll is sealed in a foil pouch under nitrogen, along with a desiccant pouch, to make a kit according to this invention.

The sealed pouch is later opened and the orthopedic casting article is removed. Using latex gloves, the roll of casting material covered with the casting aid is placed in water and squeezed twice while underwater. The roll is then taken out of the water and squeezed to remove excess amounts of water. The covered roll is used to wet both gloves being worn by rolling it between the palm and wiping both palm, transferring lubricating material from the casting aid to the gloves. The casting aid is then laid aside and the wrapping process begun.

The casting sheet is next unrolled while wrapping the sheet around the limb of a human subject to form a cast. The lubricated gloves prevent the resin from the tape from sticking to the gloves and allow the hands to move freely in the molding of the cast. The casting aid is used to re-apply more lubricant to coat the palms of the gloves during the wrapping process.

### EXAMPLE 2

A casting material is then made comprising a casting tape of fiberglass with approximately 12% polypropylene fiber, about 10 cm (4 inches) wide and 3.7 m (4 yards) long and 0.9 mm (0.36 inches) thick. The tape has approximately 25% stretch and a mesh site of approximately 40.3 holes/cm² (260 holes/inch²). The tape is impregnated with the following mixture:

| Material | % (by weight) |
|---|---|
| Isonate ™ 2143L¹ | 30.8 |
| PPG 3025² | 64.4 |
| dimorpholinodiethyl ether | 3.9 |
| benzoyl chloride | 0.1 |
| silicone | 0.2 |
| butylated hydroxytoluene | 0.2 |
| titanium dioxide | 0.4 |

¹modified diphenylmethane diisocyanate resin, sold by Dow Chemical, Midland, Michigan
²a polypropylene glycol, having a hydroxyl number of approximately 37, sold by Lyondell Chemical Company, South Charleston, West Virginia
³35,000 centipoise polydimethylsiloxane, sold by Dow Corning Corp., Midland, Michigan

The tape is made, rolled and impregnated with resin as described in Example 1.

A casting aid according to this invention is made, having the following lubricating material composition.

| Material | % (by weight) |
|---|---|
| PVP K-90¹ | 64.0 |
| Aerosol OT-75%² | 3.3 |
| W610 Ceramic Zeeospheres³ | 3.7 |
| water | 29.0 |

¹solution containing approximately 20% polyvinylpyrrolidone in water, sold by BASF
²solution containing approximately 75% sodium dioctyl sulfosuccinate in ethanol/water, sold by Cytec Industries
³ceramic microspheres, sold by 3M

The lubricating material is coated on a sheet of surface-oxidized polyethylene approximately 13 cm (5 inches) wide, 20 cm (8 inches) long, and 0.5 mm (2 mil) thick. The casting aid is then wrapped around the roll of impregnated casting sheet. The wrapped roll is sealed in a foil pouch under nitrogen, to make a kit according to this invention.

### EXAMPLE 3

A casting aid according to this invention is made, having the following lubricating material composition.

| Material | % (by weight) |
|---|---|
| PVP K-60¹ | 54.4 |
| PVP K-120² | 3.6 |
| Aerosol OT-75%³ | 6.0 |
| water | 36.0 |

¹solution containing approximately 45% polyvinylpyrrolidone in water, sold by BASF
²polyvinylpyrrolidone powder, sold by BASF
³solution containing approximately 75% sodium dioctyl sulfosuccinate in ethanol/water, sold by Cytec Industries

The lubricating material is coated on a sheet of a polyester cloth, Item 43760, sold by Guilford Mills, Greensboro, N.C. U.S.A. (The cloth has a weight of about 2.7 ounces per yard, has a caliper of about 12 mils, has about 81 courses per inch, and has a Permeation Value of about 2.2.) The sheet measures approximately 13 cm (5 inches) wide, 20 cm (8 inches) long, and 0.5 mm (2 mil) thick. The lubricant is coated on the substrate at a level resulting in a relative dry weight percentage of about 114%. The casting aid is then dried to remove substantially all moisture, in an oven at about 110° C., for about 3 hours. The casting aid is then wrapped around the roll of impregnated casting sheet, made according to Example 2. The wrapped roll is sealed in a foil pouch under nitrogen, to make a kit according to this invention.

The examples and other embodiments described herein are exemplary and not intended to be limiting in describing the full scope of compositions and methods of this invention. Equivalent changes, modifications and variations of specific embodiments, materials, compositions and methods may be made with substantially similar results.

What is claimed is:

1. A casting aid, comprising:
(a) a resin-impermeable substrate having a Permeation Value of less than about 2.8; and
(b) a lubricating material, wherein said lubricating material is coated on at least one surface of said substrate.

2. A casting aid according to claim 1, wherein said substrate is a flexible sheet, comprising a polymer selected from the group consisting of polyesters, polyolefins, and mixtures thereof.

3. A casting aid according to claim 2, wherein said polymer is polyester.

4. A casting aid according to claim 2, wherein said flexible sheet is from about 0.25 mm to about 1.0 mm in thickness, and from about 5 cm to about 13 cm in width, and from about 15 cm to about 25 cm in length.

5. A kit according to claim 1, wherein said substrate has a Permeation Value of about zero.

6. A kit according to claim 1, wherein said substrate has a Permeation Value of less than about 2.5.

7. A kit according to claim 6, wherein said substrate has a Permeation Value of from about 2.0 to about 2.3.

8. A kit according to claim 2, wherein said substrate is a non porous sheet.

9. A kit according to claim 2, wherein said substrate is a porous sheet, having from about 70 to about 90 courses per inch.

10. A casting aid according to claim 1, wherein said lubricating material comprises a lubricant selected from the group consisting of hydroxyethyl cellulose, carboxymethylcellulose, polyvinylpyrrolidone, polyvinyl alcohol, polyacrylic acid salts, polyethylene oxide polymers, and mixtures thereof.

11. A casting aid according to claim 9, wherein said lubricating material comprises a lubricant selected from the group consisting of polyvinylpyrrolidone, polyvinyl alcohol, polyacrylic acid salts, polyethylene oxide polymers, and mixtures thereof.

12. A casting aid according to claim 10, wherein said lubricant is polyvinylpyrrolidone.

13. A casting aid according to claim 10, wherein said substrate is coated with said lubricating material at a level of from about 0.002 g/cm² to about 0.005 g/cm².

14. A casting aid according to claim 10, wherein said lubricating material is coated on said substrate at a relative dry weight percentage of from about 100% to about 125%.

15. A casting aid according to claim 1, wherein said lubricating material additionally comprises an antiblocking agent.

16. A casting aid according to claim 1, wherein said lubricating material is coated on one major surface of said flexible sheet.

17. A kit for the formation of an orthopedic cast, comprising:
(a) a casting material; and
(b) a casting aid comprising a resin-impermeable substrate and a lubricating material coated on one surface of said substrate.

18. A kit according to claim 17, wherein said casting material comprises a tape impregnated with a water-activated resin composition.

19. A kit according to claim 18, wherein said resin composition comprises an isocyanate-functional prepolymer, comprising an isocyanate and a polyol.

20. A kit according to claim 19, wherein said polyol has a molecular weight greater than about 2,000, and a hydroxyl number of from about 28 to about 56.

21. A kit according to claim 19, wherein said resin composition has a level of free NCO of from about 6% to about 10%.

22. A kit according to claim 18, wherein said resin composition is essentially free of a lubricant.

23. A kit according to claim 18, wherein said tape comprises fibers selected from the group consisting of fiberglass, polyethylene, polypropylene, polyacrylonitrile copolymers and mixtures thereof.

24. A kit according to claim 17, wherein said substrate has a Permeation Value of less than about 2.8.

25. A kit according to claim 24, wherein said substrate has a Permeation Value of less than about 2.5.

26. A kit according to claim 25, wherein said substrate has a Permeation Value of about zero.

27. A kit according to claim 25, wherein said substrate has a Permeation Value of from about 2.0 to about 2.3.

28. A kit according to claim 17, wherein said substrate is a non porous sheet.

29. A kit according to claim 17, wherein said substrate is a porous sheet, having from about 70 to about 90 courses per inch.

30. A kit according to claim 17, wherein said substrate is a non-porous flexible sheet comprising polyester.

31. A kit according to claim 17, wherein said lubricating material comprises a lubricant selected from the group consisting of polyvinylpyrrolidone, polyvinyl alcohol, polyacrylic acid salts, polyethylene oxide polymers, and mixtures thereof.

32. A kit according to claim 31, wherein said lubricant is polyvinylpyrrolidone

33. A kit according to claim 17 additionally comprising a glove.

34. A kit according to claim 18, wherein said tape comprises two or more tapes formed into a roll.

35. A kit according to claim 34, wherein said roll is covered, at least in part, by said casting aid.

36. A casting aid, comprising:
(a) a resin-impermeable substrate comprising a porous fabric; and
(b) a lubricating material comprises a lubricant selected from the group consisting of polyvinylpyrrolidone, polyvinyl alcohol, polyacrylic acid salts, polyethylene oxide polymers, and mixtures thereof;
wherein said lubricating material is coated on one surface of said substrate.

37. A casting aid according to claim 36, wherein said substrate is a flexible sheet, comprising a polymer selected from the group consisting of polyesters, polyolefins, and mixtures thereof.

38. A casting aid according to claim 37, wherein said polymer is polyester.

39. A casting aid according to claim 36, wherein said flexible sheet is from about 0.25 mm to about 1.0 mm in thickness, and from about 5 cm to about 13 cm in width, and from about 15 cm to about 25 cm in length.

40. A kit according to claim 36, wherein said substrate has a Permeation Value of less than about 2.8.

41. A kit according to claim 40, wherein said substrate has a Permeation Value of less than about 2.5.

42. A kit according to claim 41, wherein said substrate has a Permeation Value of from about 2.0 to about 2.3.

43. A kit according to claim 36, wherein said substrate has from about 70 to about 90 courses per inch.

44. A casting aid according to claim 36, wherein said lubricant is polyvinylpyrrolidone.

45. A casting aid according to claim 36, wherein said substrate is coated with said lubricating material at a level of from about 0.002 g/cm² to about 0.005 g/cm².

46. A casting aid according to claim 36, wherein said lubricating material is coated on said substrate at a relative dry weight percentage of from about 100% to about 125%.

47. A casting aid according to claim 36, wherein said lubricating material additionally comprises an antiblocking agent.

48. A method of forming an orthopedic cast by hand around a body member of a human or other animal subject, using:
(a) a casting material;
(b) a casting aid according to claim 1; and
c) a glove, comprising the steps of:
(1) transferring said lubricating material to a surface of said glove;
(2) applying said casting material to said body member using said glove; and
(3) allowing said casting material to harden to form said orthopedic cast.

49. A method according to claim 48, additionally comprising a step of activating said casting material by immersing said casting material in water.

50. A method according to claim 49, wherein said casting material is immersed in water while at least partially covered by said casting aid.

51. A method according to claim 48, comprising an additional step of transferring said lubricating material to a surface of said glove performed during said step of applying said casting material to said body member using said glove.

52. A method of forming an orthopedic cast by hand around a body member of a human or other animal subject, using:
(a) a casting material;
(b) a casting aid according to claim 36; and
(c) a glove, comprising the steps of:
(1) transferring said lubricating material to a surface of said glove;
(2) applying said casting material to said body member using said glove; and
(3) allowing said casting material to harden to form said orthopedic cast.
